(19) United States
(12) Patent Application Publication
(10) Pub. No.: US 2005/0124932 A1
(43) Pub. Date: Jun. 9, 2005

(54) Venting catheter

(75) Inventors: Mark Elliott Foster, Sandy, UT (US); Sydnee F. McMillan, Sandy, UT (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 10/729,408

(22) Filed: Dec. 5, 2003

(51) Int. Cl. A61M 29/00

(52) U.S. Cl. 604/99.04; 604/910

(57) ABSTRACT

The present invention relates generally to a catheter which enables the venting or decompression of gases therethrough and, more particularly, to enteral tubes or catheters. The catheter may be configured to allow continuous venting with reduced concern about liquid leakage or the need for the user to limit their activities or assume certain positions during venting.

### BACKGROUND

Numerous situations exist in which a body cavity needs to be catheterized to achieve a desired medical goal. One relatively common situation is to provide nutritional solutions or medicines directly into the stomach or intestines. A stoma is formed in the stomach or intestinal wall and a catheter is placed through the stoma. Feeding solutions can be injected through the catheter to provide nutrients directly to the stomach or intestines (known as enteral feeding). A variety of different catheters intended for enteral feeding have been developed over the years, including some having a “low profile” relative to the patient during use and those having the more traditional or non-low profile configuration.

As indicated above, there are a variety of instances in which it may be necessary to use a catheter, one of which is the not uncommon reaction following major surgery in which a patient's stomach function is impaired for a period of time. In spite of the need to supply or supplement the body with a certain level of nutrients and the like following surgery as well as in other instances of impaired or limited gastric functionality, an unfed gut can become a source of bacteria that gets into the bloodstream. These types of problems may be resolved by the introduction of nutrients through an enteral feeding device tube properly inserted through the patient's abdominal wall, gastric wall, pylorus, duodenum, and/or into the jejunum beyond the Ligament of Treitz.

A problem universal to low profile and non-low profile devices is difficulty in decompressing or venting gases from a patient thereby enabling the release of enteric gases and resulting pressure. Just as with a patient who consumes or takes in food or medication orally without the need for enteral feeding via a catheter, a patient receiving nutrition or medication through an enteral catheter may generate gases within his enteral region either as part of the digestive process or as a result of a reaction to the medication. While some level of gas and pressure is natural in either case, a higher level of gas and pressure may develop.

Another condition where enteral catheterization and feeding may be needed is if the patient cannot swallow. The inability to swallow or otherwise satisfactorily control one's neck muscles could inhibit one's ability to relieve some of the pressure and gases which build up in the enteral region. Specifically, one may not be able to vent or expel gases naturally (e.g., belching).

Alternatively, due to a patient's condition, the patient may not be able to take a medication to reduce the gases and relieve the pressure. Furthermore, the addition of nutritional fluids or medications through a catheter that does not provide venting will result in a pressure increase inasmuch as more fluids are now present in the same area than immediately before the addition. While some of these fluids may be absorbed into the body over a period of time, the resulting pressure may be uncomfortable until some of the fluids are absorbed into the body.

In the past, an extension set was intermittently attached to the enteral feeding device to allow venting of the gases and pressures discussed above. However, that method of venting has several drawbacks. First, an extension set used to provide liquid nutritional supplement and/or medication to the enteral feeding device does not prevent liquids from passing or escaping therethrough. Although it may be possible to reduce liquid escape or back flow with the prior technique, additional effort is required to make sure that the end of the extension set is maintained at a sufficient height and that the user activity is minimized or restricted during the venting process. Second, as liquids can escape through prior catheters and extension sets and because activity of the user may need to be limited to maintain the elevation of the extension set above the enteral feeding device during the venting process, the prior techniques for venting are performed on an intermittent basis. Regardless of a patient's ability to perform the venting on their own, intermittent venting can create difficulties. For example, if not done on a regular basis it can result in discomfort to the user. However, if done on a regular basis, it may inconvenience a patient to minimize or restrict their activities and/or to need to assume certain positions several times a day to enable or facilitate venting. Furthermore, if the patient is not able to perform the necessary venting on their own, the assistance of a clinician to perform the venting may be required multiple times a day.

Accordingly, while a number of improvements have been made to conventional enteral tubes, there remains the need and the desire for a way to vent enteral feeding devices not only on a continuous basis, but such that liquids do not escape during the venting procedure, as well as the need for a venting mechanism and procedure which do not restrict the activity or position of a user while the venting is being performed. The present invention meets these needs.

### SUMMARY OF THE INVENTION

In response to the difficulties and problems discussed above a venting catheter has been developed. More specifically, one aspect of this invention is directed to a catheter adapted for insertion into a cavity, the catheter having a first lumen, a second lumen, and a venting mechanism adapted to allow for the release of pressure from the cavity. The catheter also may have a retention member adapted to retain the catheter in the cavity. The venting mechanism may be configured so as to allow gases but not liquids to pass therethrough.

Another aspect of the present invention is directed to a catheter which has the ability to allow venting of a cavity into which the catheter is inserted. The catheter may generally include a first lumen, a second lumen, a third lumen, and a venting mechanism, wherein one of the lumens is a venting lumen, and another is an inflation lumen. The adapter is desirably configured so as to allow gases but not liquids to pass through the venting mechanism thereof.

The present invention also is directed to a balloon catheter having a first lumen and a second lumen; a mechanism capable of at least partially blocking the flow of liquids through the second lumen; an inflation lumen; and a balloon member. The balloon member is in fluid communication with the inflation member and is adapted to retain the catheter in a body cavity. The catheter is adapted to allow for the release of pressure from a cavity into which the catheter can be inserted.

The invention will be more fully understood and further features and advantages will become apparent as reference is made to the following detailed description of exemplary aspects of the invention and the accompanying drawings.

### BRIEF DESCRIPTION OF THE DRAWINGS

The above and other objects, features and advantages of the invention will become apparent from consideration of the subsequent detailed description presented in connection with the accompanying drawings in which:

FIG. 1 is a perspective view of one aspect of the present invention;

FIG. 2 is cross-sectional view of one aspect of the present invention, the catheter having a venting mechanism positioned therein;

FIG. 2A is an enlarged view of the portion of the catheter in FIG. 2 within the hashed circle;

FIG. 3 is a cross-sectional view of another aspect of the present invention, the catheter having three lumens;

FIG. 3A is an enlarged view of the portion of the catheter in FIG. 3 within the hashed circle;

FIG. 4 is a cross-sectional view of another aspect of the present invention; and

FIG. 5 is a cross-sectional view of an alternate aspect of the present invention.

### DETAILED DESCRIPTION OF THE PRESENT INVENTION

Reference will now be made to the drawings in which the various elements of the present invention will be given numeral designations and in which the invention will be discussed so as to enable one skilled in the art to make and use the invention.

The present invention relates generally to an apparatus which enables the venting or decompression of gases therethrough and, more particularly, to enteral tubes or catheters.

It will be appreciated that throughout the disclosure reference is made to enteral feeding catheters for purposes of ease of reading and understanding the disclosure; however, the present invention is not intended to be limited to enteral feeding devices, enteral feeding tubes or the like.

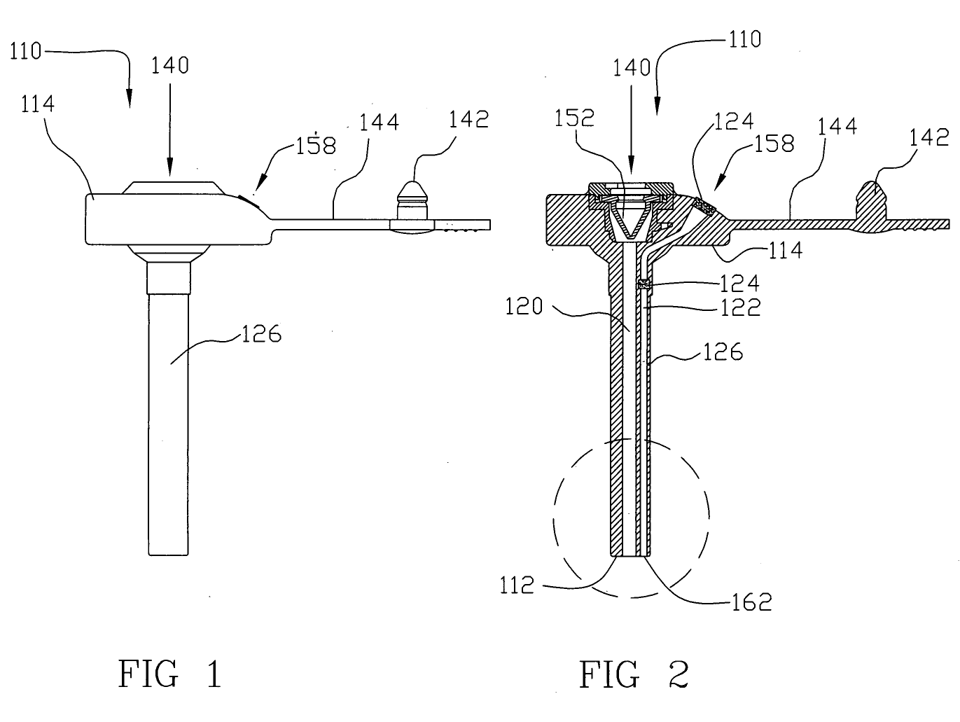

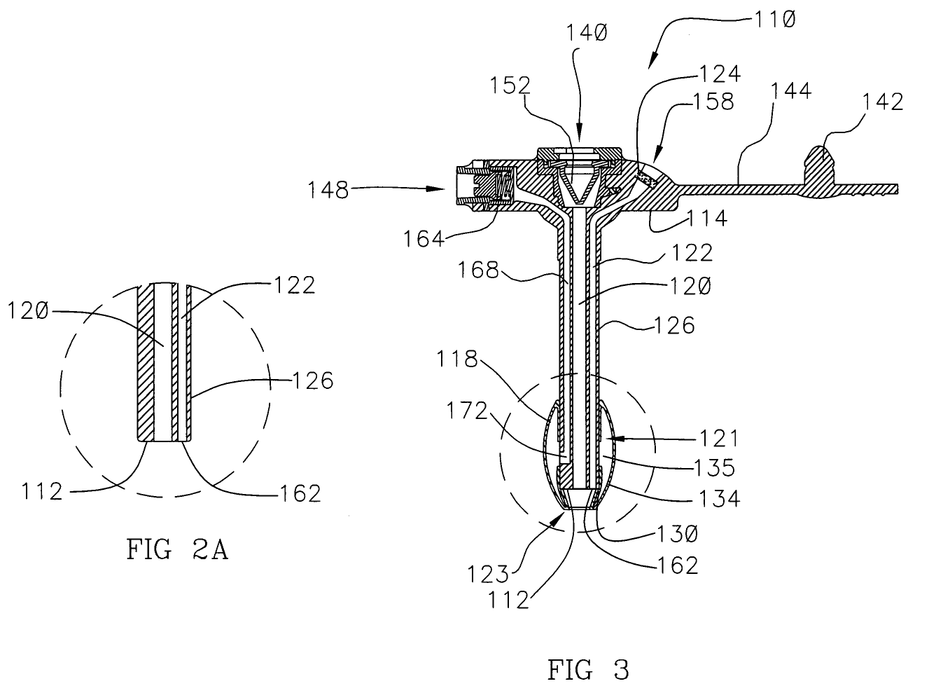

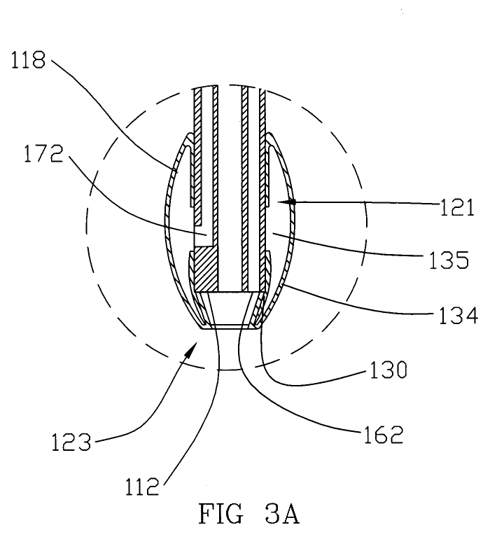

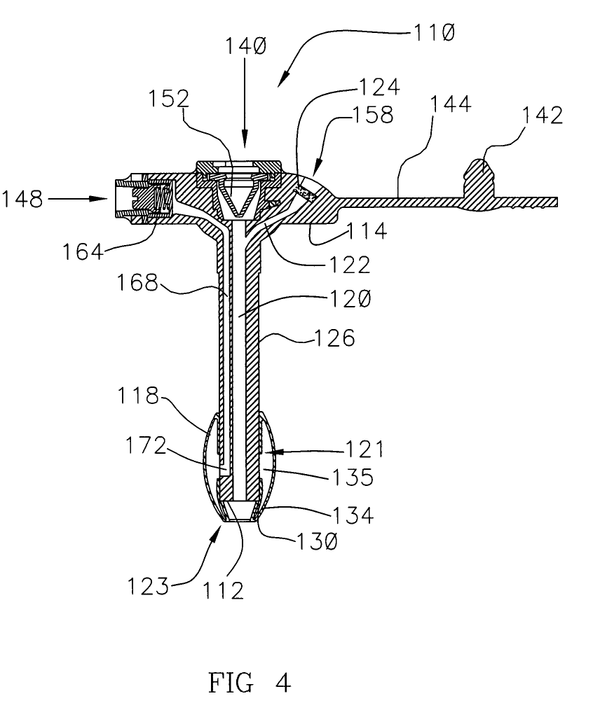

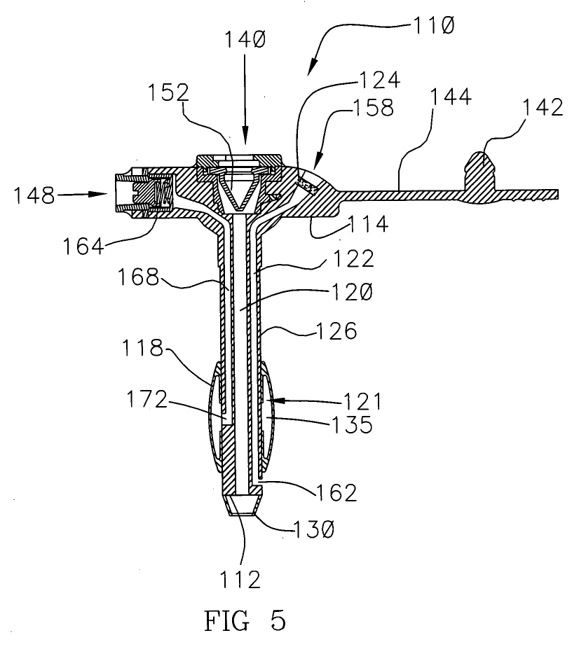

Referring now to FIGS. 1-3A, there is shown a first catheter (FIGS. 1-2A) and a second catheter (FIGS. 3 and 3A) made in accordance with the teachings of the present invention. The catheters 110 generally include a head 114 (FIGS. 1, 2 and 3) and a catheter shaft 126 (FIGS. 1-3A). The head 114 has a proximal opening 140 (FIGS. 1, 2 and 3) to a feeding lumen 120 (FIGS. 2-3A) within the shaft 126, for bolus feeding or providing other nutrient fluids, formula, and the like to a patient (not depicted). The catheter 110 also shows an optional stiff tip 130 (FIGS. 3 and 3A) attached at the distal end 112 of the catheter shaft 126. The stiff tip 130 (FIGS. 3 and 3A) has an interior surface 131 (FIG. 3A) which defines a passageway which is configured for the passage of fluids, solutions, certain solids, or the like therethrough and into or out of the catheter 110. An anti-reflux valve 152 (FIGS. 2 and 3), which is generally included to prevent back-flow of the nutrient formula, is shown disposed between the opening 140 (FIGS. 1, 2 and 3) and the feeding lumen 120 (FIGS. 2-3A). A second opening or port 158 (FIGS. 1, 2 and 3) is shown disposed in the head 114 (FIGS. 1, 2 and 3) and communicates with a second lumen 122 (FIGS. 2-3A) which extends generally longitudinally through the shaft 126. The second lumen 122 is shown terminating at the distal end 112 of the catheter shaft 126 but, alternatively, could terminate into the first lumen 120 (as illustrated in FIG. 4). Another option is for the second lumen to terminate laterally to the shaft 126 at port 162 (FIG. 5) depending on the configuration of the catheter (so long as port 162 is positioned sufficiently close to distal end 112 such that the port 162 is within the cavity to be vented when the catheter is properly positioned). The catheter 110 also includes a venting mechanism 124, shown in FIGS. 2, 3, 4 and 5, as being within the second lumen 122.

Also shown associated with the head 114 (FIGS. 1, 2 and 3) is a plug 142 (FIGS. 1, 2 and 3) for the proximal opening 140 (FIGS. 1, 2 and 3) and a lanyard 144 (FIGS. 1, 2 and 3) for retaining the plug 142. The plug 142 can be inserted in the opening 140 thereby reducing or precluding contamination whenever the opening 140 is not in use. Openings 148 (FIG. 3) and 158 (FIGS. 1, 2 and 3) can be sized so as to be capable of receiving plug 142; moreover, plugs of other sizes adapted to be inserted into openings 148 and 158 could be included on lanyard 144. Feeding lumen 120 (FIGS. 2-3A) extends longitudinally through shaft 126 and is shown terminating at the distal end 112 (FIGS. 2-3A) of the shaft 126 although other termination points (e.g., lateral termination through an opening or openings (not shown) created in side of the catheter shaft 126) are contemplated in other catheter configurations.

FIGS. 2 and 2A differ from FIGS. 3 and 3A in that the number of lumens in the catheters are two and three, respectively. While there need only be two lumens, one of which is used to assist in venting, it will be appreciated that the number of lumens in a catheter is not otherwise limited in concept, although a particular catheter may be limited because of size or shape as to the number of additional lumens it may contain.

Common reasons for the inclusion of more than two lumens include but are not limited to: 1) the use of a first feed lumen for a first region (e.g., the gastric cavity), a second feed lumen for a second region (e.g., the jejunal region), and another lumen for venting, and/or 2) the use of a first feed lumen, another lumen for venting, and another lumen (e.g., an inflation lumen) to communicate with a retention member (e.g., a balloon member).

While not essential to the catheter 110, many aspects of the present invention also will include a retention member (shown as a balloon member 118 in FIGS. 3 and 3A) capable of retaining the catheter 110 in the stoma of a patient. It will be appreciated that any suitable retaining member is contemplated by the present invention. The term retention member is intended to mean or include but is not limited to a balloon or balloon member, a sleeve, an inflatable or expandable member, an elastomeric sleeve, an expandable region or portion, a unitary component (i.e. combination tip and balloon member as discussed in U.S. patent application Ser. No. 10/306,999 (Attorney Docket No. 17,110A) and Ser. No. 10/306,994 (Attorney Docket No. 17,110C)), any other suitable expandable means, or the like.

It will be appreciated that throughout the disclosure reference is made to inflation of the retention member and, more specifically, the balloon member, however, the present invention is not intended to be limited only to inflation. That is, while inflation is used herein for purposes of ease of reading and understanding the disclosure, the term inflation also is intended to mean or include but is not limited to expansion, enlargement, swelling or the like.

Referring again to FIGS. 3-3A, an opening 148 (shown as an inflation port in FIG. 3) is shown as being disposed in the head 114 and in communication with inflation lumen 168 which extends longitudinally through the shaft 126. The inflation lumen 168 is shown terminating laterally to the shaft 126 at port 172 into the cavity 135 created by the balloon member 118 and the shaft 126.

A one-way valve 164 (FIG. 3) may be disposed between the inflation port 148 and inflation lumen 168. Application of positive fluid pressure, such as with air or saline, within and/or upon the inflation lumen 168 by way of the inflation port 148 (FIG. 3) may cause the balloon member 118 to inflate as the fluid fills the cavity 135. Valve 164 (FIG. 3) helps prevent inadvertent deflation of the balloon member 118.

It will be appreciated that the size and shape of the cavity 135 defined by or between the exterior of the shaft 126 and the balloon member 118 may be varied during production or controlled by the user or clinician during use. Additionally, as discussed in more detail below, the balloon member 118 of the catheter 110 may be designed to have a certain size and/or shape in either or both of its inflated or uninflated configurations. It will be understood and appreciated that varying the length of the balloon member 118 and/or the points along the shaft 126 at which the ends 121 and 123 of the balloon member 118 are attached may affect the shape of the resulting balloon member.

The various components of balloon catheter 110 may be made of any suitable material and desirably may be formed from bio-compatible materials such as medical grade silicone or the like. While valves 152 (FIG. 3) and 164 (FIG. 3) may be formed of any suitable material, they are desirably made of a suitable polymer such as polycarbonate.

Depending on the size and shape of the body cavity in which the catheter 110 is inserted as well as the nature of material to be passed therethrough the size of the catheter 110 as well as the inflated and/or uninflated length of the balloon member 118 may be varied. That is, in some instances, it may be desirable to use catheters having larger and/or wider shafts than in other aspects. Additionally, the balloon member 118 of the catheter 110 may be designed to have a certain size and/or shape in either or both of its inflated or uninflated configurations.

The catheter 110 may be of a “low profile” as shown in FIGS. 1, 2 and 3 such that the head 114 may rest on or near the skin of a patient (not shown) when the catheter 110 is properly positioned within the patient. Or, the catheter may take a more traditional or non-low profile configuration (such as that discussed in U.S. Pat. No. 4,701,163 to Parks) wherein the head of the catheter generally extends away from the skin of the patient when the catheter is properly positioned in the patient and wherein the head is frequently larger as concealment or the potential therefore may not be of significant concern.

In all prior traditional or low profile catheters extension sets (not shown) were necessary to achieve the desired or necessary venting. With a significant length of tubing associated therewith, the extension set adds considerable bulk to the catheter, making concealment of the catheter and its components more difficult and troublesome. This is especially true with the use of a low profile catheter as the portion of the catheter which extends above the patient's skin is generally minimal and does not include much tubing external to the patient so as to conceal the presence of the catheter as well as to reduce the likelihood that the device will get caught on or in something and potentially dislodge the catheter from the patient. The present invention provides a solution which overcomes problems and difficulties associated with the prior catheters.

As suggested above, the present invention is intended to work with a variety of enteral feeding devices. Exemplary enteral feeding devices include, but are not limited to, gastrostomy devices, jejunostomy devices, transgastric jejunal devices and the like any of which may be low profile or non-low profile. A more detailed description and discussion of specific aspects of suitable catheters may be found in U.S. Pat. No. 5,997,503 to Willis et al. and U.S. Pat. No. 5,997,546 to Foster et al., and commonly assigned copending U.S. patent application Ser. No. 10/159,514 (Attorney Docket No. 17,508A), filed May 31, 2002.

Even with the advantages each of the catheters above exhibit over other available catheters, very few prior catheters have provided for venting of any sort. However, even those with some venting ability have demonstrated at least one of two drawbacks. Namely, prior venting catheters have an open lumen which does not restrict the flow of fluids therethrough and thus allows for undesirable liquid leakage from the cavity and/or the prior catheters have required the insertion of an external device, frequently an extension set or the like, to open a valve in a lumen that is intended to pass liquids therethrough to the patient. Again, in either instance the potential for liquids to leak from a prior catheter is present, and in some instances the catheters require insertion of a large external device which may need to be left in place for a period of time thereby potentially limiting the activities of the user, the position of the user, and/or the ability of the user to conceal the presence of the catheter and its components. The present invention provides a solution which overcomes problems and difficulties associated with the prior catheters.

Having discussed the catheters of the present invention in general terms the discussion hereof now shifts to a more detailed discussion of some of the venting mechanisms of the present invention. Referring again to FIG. 3, there is shown a catheter 110 having a first lumen 120, a second lumen 122, and a venting mechanism 124 adapted to enable venting from a cavity (not shown) into which the catheter 110 may be inserted. Although not required in all aspects of the present invention, the catheter 110 is shown having a balloon member 118 connected with a third or inflation lumen 168 so as to allow for the expansion and contraction of the balloon member 118. In this aspect of the present invention, the second lumen 122 is a venting lumen in that the second lumen 122 is intended to provide a way of allowing gas to vent from the cavity (not shown) in which the catheter 110 is inserted. Specifically, the second lumen 122 is shown having the venting mechanism 124 provided therein. Although illustrated in the head 114 of the catheter in FIG. 3, exemplary alternative positioning of the venting mechanism is shown in FIGS. 2, 4 and 5.

It will be appreciated that some catheters will have different configurations which may affect the ability or desirability of a lumen to be a venting lumen; however, solely for ease of disclosure and discussion herein, the second lumen has been selected as the venting lumen. It will be recognized that in other aspects of the present invention a first, third or other lumen could be a venting lumen.

It will be further appreciated that the type of venting mechanism used in a particular catheter may vary depending on the intended use of the catheter 110. For example, if the second or venting lumen 122 is intended to allow for the transmission of fluids from the head 114 of the catheter 110 to the distal end 115 thereof, one type of venting mechanism (e.g., a butterfly valve, a valve having position or orientation sensitive properties (i.e., a gravity controlled ball valve), or the like) may be selected, while another type of venting mechanism (e.g., a gas permeable membrane) may be selected if only gases are to pass through the venting mechanism. As suggested herein, the venting mechanism 124 which provides for or allows venting to occur through the lumen 122 may be located in the head of a catheter 110, at either end of the venting lumen 122 or the venting mechanism 124 may be within the lumen 122. The location of the venting mechanism may be selected in part based on the type of venting mechanism included. Multiple venting mechanisms in one aspect of the present invention are also contemplated.

It will be appreciated that different types of venting mechanisms may enable operation (passive and/or active) under different operating conditions. For example, if a gas permeable and liquid impermeable membrane such as that shown at 124 in FIG. 3 is used, the catheter 110 may be able to vent continuously provided, of course, that there is not another limiter in place such as a port plug 142 (FIGS. 1, 2, and 3).

Alternatively, other mechanisms may be used which restrict the conditions under which venting may occur or what must occur to begin or limit venting. For example, one aspect of the present invention contemplates a venting mechanism that is triggered or operated remotely. Such remote operation allows for discrete operation of the venting mechanism and/or the operation of the mechanism by a third party. Another aspect of the present invention contemplates a venting mechanism that is triggered to open whenever the venting mechanism and/or catheter become exposed to certain conditions (e.g., the existence of certain pressure levels within the cavity). Yet another catheter of the present invention may include a button or other trigger to activate the venting mechanism. In this instance, the term activation is intended to include, but not be limited to, opening, closing, or triggering an opening or closing at a later time. It is contemplated that such a trigger or activation could be used in a variety of ways, including, for example, to open the venting mechanism and have it remain open until the trigger or activating mechanism is released, to open the venting mechanism and have it remain open until triggered or activated again, to open and/or close the venting mechanism after a specific amount of time has elapsed or upon the exposure to certain conditions, to close and then subsequently open the venting mechanism after the elapse of a period of time or upon the exposure to certain conditions.

Another aspect may include a venting mechanism that is opened upon the occurrence of a certain event, or activation or trigger, or exposure to certain conditions, but will gradually close over a period of time (e.g., a venting mechanism made out of a memory material). Still another catheter of the present invention may include a device such as a gravity operated ball valve which allows venting to occur when the catheter is in certain orientations which generally correspond to when a user is in certain positions (i.e., standing, lying on their back) but not in others (lying on their stomach). Further, such a device also may be configured to allow partial venting if the patient lies on their side. For example, if a patient were to lie on their back, the device may be fully open to venting; but, as the patient rolls onto their side, the device would cause the lumen to be partially closed off, and the more the patient moved towards being on their stomach (facing downwards), the more the lumen would be closed off. It is appreciated that such a device could be configured so that lumen could be closed off at any catheter orientation less than requiring the patient to face downward. The importance of closing off the lumen may lessen if the mechanism is limited only to the ventilation of gases rather than being an open lumen and allowing for the passage of liquids.

It will be appreciated that while a variety of venting mechanisms are discussed herein, including those which are described as continuously or always open (at least partially), the catheters of the present invention may also include a second mechanism for obstructing or otherwise limiting the venting ability of the catheter irrespective of the first or main venting mechanism. For example, a catheter of the present invention may include a plug 142 or the like, such as that shown in FIGS. 1, 2, and 3, which may be inserted into a lumen or port therefor so as to restrict or completely block the ability of the lumen to vent. Such a plug or the like may be included, for example, where it is generally desirable to have constant or continuous venting or at least the ability therefore, but that there may be a limited number of times or places that venting would be unwanted (e.g., swimming, showering or other water activities, as well as functions or gatherings where the odor of venting gases potentially could be unpleasant or even offensive to others present in the room). Alternatively, any suitable combination of two or more venting mechanisms and/or venting limiters (e.g., plug 142) may be used in conjunction with each other to combine the features of each into one catheter. For example, a gas permeable membrane could be incorporated into a catheter having a manually triggerable valve or vent, so that when the valve is triggered or activated gas or fluids may pass therethrough but that only gases will pass through the membrane and out of the catheter. Alternately, the membrane could be located closer to the distal end 112 of the catheter 110 so as to only allow gases to approach the valve from the distal end of the catheter.

Referring again to FIGS. 2 and 3, the specific venting mechanism illustrated is a gas permeable membrane 124. The membrane may be such that it permits gases but not liquids to pass therethrough so as to allow for a venting or decompression of gases in a cavity (not shown) in which the catheter 110 is inserted. It will be recognized that a variety of suitable membranes may be selected for inclusion in a catheter in accordance with the present invention. For example, a membrane which permits gas to diffuse or permeate therethrough at a certain rate may be chosen in certain aspects of the invention, while a membrane having different flow rate characteristics may be selected in another catheter. Further, depending on the intended use of the catheter, the catheter may be exposed to certain gases whose flowthrough may be better able to be regulated with a membrane having a specific makeup.

As suggested above, venting mechanism 124 (e.g., the membrane) may exemplarily be located in the head 114 of the catheter 110 (see FIG. 3), at the distal end 112 of the catheter 110, in the catheter shaft 126 (see FIGS. 2, 4 and 5), or even outside or over the port 158 of the second or venting lumen 122 (see FIG. 2). It will be appreciated that the location of the venting mechanism may be selected or determined in part based on the type of venting mechanism included.

Depending on the type of venting mechanism included in the catheter as well as the location and manner in which it is secured within or to the catheter, removal and replacement of at least a portion of the venting mechanism is contemplated so as to allow for extension of the usable life of the catheter. However, the design of certain aspects of the present invention may prevent removal and/or replacement of the venting mechanism.

For example, if the venting mechanism is an insert, such as one comprising at least in part a porous material, the functional life or length of efficaciousness of the insert may be less than that of the remaining catheter components. It will be appreciated that a variety of venting mechanisms could be in the form of an insert that may be a replacement for a pre-existing venting mechanism or a post-manufacture addition. Any suitable insert is contemplated by the present invention. An exemplary insert including at least in part a porous material may include reticulated polymer foams, expanded polymers (such as Porex expanded polymers available from Porex Corporation, having offices in Fairburn, Ga.), expanded PTFE (such as Gore-Tex® expanded PTFE available from W.L. Gore & Associates, Inc. having offices in Newark, Del.), porous metals, and powdered metals or the like.

As noted herein, any suitable catheter incorporating a venting mechanism of the present invention is contemplated by the present invention, including those catheters having more than two lumens. More specifically, at least one aspect of the present invention contemplates a catheter having a first lumen, a second lumen, a third lumen, and a venting mechanism, wherein one of the lumens is a venting lumen, and another of the lumens is an inflation lumen. The catheter may also include a retention member adapted to retain the catheter in a cavity into which the catheter may be inserted. As above, any number of suitable retention members are contemplated, however, a balloon member or a component incorporating a balloon member are most common and are generally in communication with an inflation lumen.

Another aspect of the present invention is directed to a balloon catheter having a first lumen, a second lumen, a mechanism capable of at least partially blocking the flow of liquids through the second lumen, and an inflation lumen and a balloon member adapted to retain the catheter in a body cavity. As will be appreciated from the discussion herein, any number of suitable mechanisms may be incorporated. Suitable mechanisms include but are not limited to a gas permeable venting mechanism or a gas permeable membrane. The mechanism may be such that in one position it allows fluids to pass through the second lumen and at least partially blocks the flow of liquids through the second lumen in another position. It will be appreciated that the mechanism may be such that the orientation of the catheter effects the ability of fluids to pass through the mechanism. The mechanism may be such that it is gas permeable, but liquid impermeable and thereby prevents liquids from being vented through the mechanism.

It should be appreciated that each example and drawing is provided by way of explaining the invention, and not as a limitation of the invention. For example, features illustrated or described with respect to one aspect may be used with another aspect to yield still a further aspect of the invention. These and other modifications and variations are within the scope and spirit of the invention.

It should further be appreciated that each aspect of the present invention may not possess each and every component described or contemplated hereby and/or may not possess each and every advantage described or contemplated herein but all such aspects are nevertheless contemplated to be within the scope of the disclosure and the attached claims.

Each of the patents, applications, and/or references mentioned, referred to, or discussed herein is herein incorporated by reference in its entirety.

While various patents and other reference materials have been incorporated herein by reference, to the extent there is any inconsistency between incorporated material and that of the written specification, the written specification shall control. In addition, while the invention has been described in detail with respect to specific aspects thereof, those skilled in the art, upon obtaining an understanding of the invention, may readily conceive of alterations to, variations of, and equivalents to the described aspects. It is intended that the present invention include such modifications and variations as come within the scope of the appended claims and their equivalents.

1. A catheter adapted for insertion into a cavity, the catheter comprising:
a first lumen;
a second lumen; and
a venting mechanism adapted to allow for the release of pressure from the cavity.

2. The catheter of claim 1 wherein the catheter further comprises a retention member adapted to retain the catheter in the cavity.

3. The catheter of claim 1 wherein the venting mechanism is gas permeable and liquid impermeable.

4. The catheter of claim 1 wherein the venting mechanism is continuously open.

5. The catheter of claim 1 wherein the venting mechanism is open, partially open, or closed.

6. The catheter of claim 1 wherein the venting mechanism is a gas permeable and liquid impermeable membrane.

7. The catheter of claim 1 further comprises a head positioned at one end of the first and second lumens, and wherein the venting mechanism is located in or about the head of the catheter.

8. The catheter of claim 1 further comprises a head positioned at one end of the first and second lumens, and wherein the venting mechanism is located distal the head of the catheter.

9. The catheter of claim 1 wherein the venting mechanism is located at one end of one of the lumens.

10. The catheter of claim 1 wherein the venting mechanism is capable of being remotely actuated.

11. The catheter of claim 1 wherein the venting mechanism is a butterfly valve.

12. The catheter of claim 1 wherein the venting mechanism is a gravity operated ball valve.

13. The catheter of claim 1 further comprises a third lumen.

14. The catheter of claim 13 wherein the third lumen is an inflation lumen.

15. The catheter of claim 1 wherein the catheter is a low profile catheter.

16. The catheter of claim 1 further comprises a trigger to operate the venting mechanism.

17. The catheter of claim 2 wherein the retention member is a balloon member.

18. The catheter of claim 2 wherein the retention member is a unitary component.

19. The catheter of claim 1 further comprises a second mechanism adapted to further control the venting ability of the catheter.

20. The catheter of claim 1 wherein the catheter is an enteral feeding catheter.

21. The catheter of claim 1 wherein the venting mechanism is an insert comprising at least in part a porous material.

22. The catheter of claim 21 wherein at least a portion of the porous material of the insert is selected from the group consisting of reticulated polymer foams, expanded polymers, expanded PTFE, porous metals, and powdered metals.

23. The catheter of claim 21 wherein at least a portion of the venting mechanism is removable from the catheter.

24. A catheter comprising:
a first lumen;
a second lumen;
a third lumen; and
a venting mechanism;
wherein one of the lumens is a venting lumen, and another of the lumens is an inflation lumen.

25. The catheter of claim 24 wherein the catheter further comprises a retention member adapted to retain the catheter in a cavity.

26. The catheter of claim 24 wherein the venting mechanism is gas permeable.

27. The catheter of claim 24 wherein the venting mechanism is a gas permeable and liquid impermeable membrane.

28. The catheter of claim 24 wherein the venting mechanism is capable of being remotely actuated.

29. The catheter of claim 24 wherein the venting mechanism is a butterfly valve.

30. The catheter of claim 24 wherein the venting mechanism is a gravity operated ball valve.

31. The catheter of claim 24 wherein the catheter is a low profile catheter.

32. The catheter of claim 24 further comprising a trigger to operate the venting mechanism.

33. The catheter of claim 24 wherein the retention member is a balloon member.

34. The catheter of claim 24 wherein the retention member is a unitary component.

35. The catheter of claim 24 further comprises a second mechanism adapted to further control the venting ability of the catheter.

36. The catheter of claim 24 wherein the catheter is an enteral feeding catheter.

37. A balloon catheter comprising:
a first lumen and a second lumen;
a mechanism capable of at least partially blocking the flow of liquids through the second lumen; and
an inflation lumen; and a balloon member; wherein the balloon member is in fluid communication with the inflation member and is adapted to retain the catheter in a body cavity; wherein the catheter is adapted to allow for the release of pressure from a cavity into which the catheter can be inserted.

38. The catheter of claim 37 wherein the mechanism is a gas permeable venting mechanism.

39. The catheter of claim 37 wherein the mechanism is a gas permeable and liquid impermeable membrane.

40. The catheter of claim 37 wherein the mechanism at least partially blocks the second lumen based on the orientation of the catheter.

41. The catheter of claim 37 further comprises a retention member.

42. The catheter of claim 37 further comprises a second mechanism adapted to further control the venting ability of the catheter.
